(19) United States
(12) Patent Application Publication
(10) Pub. No.: US 2010/0129472 A1
(43) Pub. Date: May 27, 2010

(54) Compositions And Method For The Treatment Of Acne

(76) Inventor: Daniel Pry, Deming, WA (US)

Correspondence Address: Michel Labadie, 210 - 15252 32 AVE, SURREY, BC, V35 OR7, CA

(21) Appl. No.: 12/502,533

(22) Filed: Jul. 14, 2009

Related U.S. Application Data
(60) Provisional application No. 61/080,599, filed on Jul. 14, 2008.
(60) Provisional application No. 61/102,713, filed on Oct. 3, 2008.

(51) Int. Cl. A61K 33/04; A61K 33/06; A61K 36/02; A61K 36/752; A61K 36/886; A61K 36/53; A61K 36/63; A61K 36/76; A61P 17/10; A61K 36/355

(52) U.S. Cl. 424/684; 424/705; 424/195.17; 424/736; 424/744; 424/745; 424/769; 424/775

(57) ABSTRACT

The present invention relates to methods for the treatment and reduction of acne and compositions comprising sulfur colloid and a 100% natural base.

### FIELD OF THE INVENTION

This invention relates to a formulation for the treatment of acne. Specifically, the invention relates to an acne cream formulation comprising sulfur colloid.

### BACKGROUND OF THE INVENTION

Acne vulgaris (commonly called acne) is a skin disease caused by changes in the pilosebaceous units (skin structures consisting of a hair follicle and its associated sebaceous gland). Severe acne is inflammatory, but acne can also manifest in noninflammatory forms. Acne lesions are commonly referred to as pimples, spots, or zits.

Acne is most common during adolescence, affecting more than 85% of teenagers, and frequently continues into adulthood. For most people, acne diminishes over time and tends to disappear, or at the very least decrease, after one reaches his or her early twenties. There is, however, no way to predict how long it will take to disappear entirely, and some individuals will continue to suffer.

There are a variety of treatments for acne but many are either invasive or are not clinically proven. Moreover, available acne preparations commonly contain sulfates, parabens, colorants, fragrances that exacerbate such condition.

Sulfur by itself or in combination with synthetic chemical formulas has been utilized since the 1950's as an effective FDA approved OTC Acne medication. Since then, no formulator has been able to create a 100% Natural base to house Sulfur. Consequently, all products currently marketed with Sulfur have non-natural bases and tend to include sulfates, parabens, fillers, colorants and fragrances.

There is a need for holistic alternatives that are 100% organic/natural formulation comprising sulfur colloid.

### SUMMARY OF THE INVENTION

The present invention focuses on the fundamental causes of Acne, the primary characteristics of healthy skin, and the use of earth-based ingredients. The present invention provides a formulation that contains 100% organic/natural formulation. Specifically, the present invention relates to methods for the treatment and reduction of acne and compositions comprising sulfur colloid and a 100% natural base.

Further objectives and advantages will become apparent from a consideration of the description, drawings, and examples.

### DETAILED DESCRIPTION

In the following description, specific terminology is employed for the sake of clarity. However, the invention is not intended to be limited to the specific terminology so selected. It is to be understood that each specific element includes all technical equivalents which operate in a similar manner to accomplish a similar purpose. Each reference cited here is incorporated by reference as if each were individually incorporated by reference.

The present invention is directed to compositions and methods for the treatment of acne. In one embodiment of the invention, the formulation contains sulfur colloid with a 100% natural base including a synergistic combination of absorbent and adsorbent clays, fillers with micro-nutrient rich herbs and minerals, vegetables or fruits.

In another aspect, the invention is directed to methods of stabilizing and homogenizing the formulations to provide consistency with quality assurance guidelines.

The present invention utilizes unique 100% natural acne formulations for specific functions for the treatment of acne. These unique formations of the present invention cleanse and provide an antibacterial function without any use of soaps, sulfates or synthetic chemicals. In one embodiment, the formulation does not contain, is free of or contains a minimal amount of soaps, sulfates, detergents, colorants or fragrances.

In a further embodiment of the invention, the active ingredient may comprise sulfur colloid in an amount from about 5-7% weight/weight (“w/w”) of the total final composition. In a particular embodiment, the amount can be from about 1-50%, 3-25%, 5-10%, 5-7%, or 5%, 6%, or 7%.

In another embodiment, the formulation comprises water in an amount from about 54-56% w/w of the total final composition.

In a further embodiment, the formulation may comprise an adsorbent cleansing agent. The adsorbent cleansing agent may comprise a clay or blend of clays. Embodiments of clays include Fullers Earth, Kaolin clay, Bentonite clay and Xanthan Gum powder.

In another embodiment, the formulation may comprise plant juices and extracts.

In another embodiment, the formulation may comprise an anti-inflammatory agent such as Arnica Montana Flower Extract, Chamomile, Lavandula Angustifolia (Lavender) Oil or Hemp Seed oil.

In another embodiment, the formulation may comprise an antibacterial or antifungal agent such as raw, organic sources of Origanum Vulgare (Oregano) Leaf Extract, Tea Tree, Hydrastis canadensis (Golden Seal) Root Extract, Olea europaea (Olive) Leaf Extract or other compatible components that display significant topical efficacy in reducing bacteria count and reducing the infection rate of Acne Sufferers.

The formulation may further comprise Salix Alba (Willow) Bark Extract (White Willow Bark), Cananga Odorata (Ylang Ylang) Flower Oil, Medica Limonum (Lemon) Peel Oil, Citrus Aurantium Bergamia (Bergamot) Fruit Oil, Rosmarinus Officinalis (Rosemary) Leaf Extract, Thymus Vulgaris (Thyme) Extract, Calendula Officinalis Flower Extract, Witch Hazel, or Grapefruit Seed.

The composition disclosed herein may be a formulation, cleanser, cream, foam, mask, toner, ointment, or solution for topical use for human skin conditions.

In another embodiment, the invention is for topical application, comprising, an active ingredient and a cosmetically acceptable vehicle. In a particular embodiment, the invention is directed to a topical composition for the treatment of acne. In another embodiment, the invention is a composition for reducing and relieving an individual from contracting acne. In yet a further embodiment, the composition is effective at reducing the frequency and intensity of recurring acne lesions and redness. In another embodiment, the invention is directed to compositions for providing skin brightening, scar reduction, blood circulation, and reducing inflammation.

In one embodiment, the formulation may contain a clay, herb, and sulfur ingredient. In another embodiment, the formulation may contain pure plant juice and sulfur ingredient.

In another exemplary embodiment, the invention relates to methods of treating acne comprising administering a topical formulation. In yet a further embodiment, the invention is directed to a method of treating acne in a human in need thereof comprising administering a topical composition comprising sulfur colloid and a 100% natural base.

The formulation may be applied daily, weekly or monthly. The formulation may be applied once, twice or up to about five times a day.

In another embodiment, one or more formulations are combined and used on a daily basis, e.g., one during the day and one at night, or over a period of time. In another embodiment, the formulation is a spot treatment system wherein one formula is applied at one point during the day and the other formulation is applied at another time during that same day.

In another embodiment, the invention relates to a kit or a container with a label and comprises a formulation, a baggie, cotton balls or pads, or a doe-foot wand and instructions for use. In another embodiment, the formulation is applied using a doe-foot wand.

In another embodiment, the invention relates to a method of producing a formulation as set forth in Examples 1 or 2.

The formulas and methods described herein can be used complimentary to, or successively of, each other. Additionally, all can be effectively utilized independently of each other.

### Example 1

1. Clay/Herb/Sulfur Cleanser/Mask Combination: Soap/Sulfate/Detergent-free 100% Natural OTC formula that is a daily cleanser, as well as a 3 times a week mask. All similar products in this category utilize sulfates or surfactants of some form. This one product can be used by itself, or in combination with the following products. An effective blend of clays complimented with an herbal cocktail can be used as a one-item-only approach for acne—also a unique concept within this category. No system or steps are required for effective results. The blend of clays and herbs, listed in formulas, creates an adsorbent cleansing medium whose mechanism of cleansing the skin does not emulsify dirt, debris and toxins. Rather, it adsorbs debris, dead skin cells, toxins, bacteria, fungus. The former are all potential irritants that exacerbate the Acne condition. Washing the skin complexion with soap, not only strips with skin, it also leaves behind residues that can intensify an Acne outbreak. This combination of clays and herbs effectively cleans the skin without stripping the acid mantle or unbalancing the lipid barrier of the skin—both of which are totally removed by sulfates in any form. Proper maintenance of the Acid mantle and Lipid barrier are quintessential for healthy skin. This formulation helps maintain and improve both.
2. Pure Plant Juice/Sulfur Toner: This 100% Natural pure plant solution is a topically applied aqueous medium that is dispensed into a cotton ball or pad then sparingly swept over the entire complexion and left on the skin without rinsing. It is used throughout the day as needed to control excess oil production, remove waxy buildup in the pores, condition the skin, keep skin shine-free and remove environmental toxins. It may be effectively applied 1-5 times per day. This is unique in that standard Acne toners are typically used after cleansing only and cannot be applied throughout the day. This product can be tossed in a baggie with a few cotton balls or pads and used throughout the day to improve skin's condition and address follicular build-up as it occurs. Additionally, this formulation does not require a cleansing stage prior to use. Positive results are experienced with the use of this formulation independent of other products. The function with this aqueous solution utilizes sulfur and additionally Aloe, White Willow Bark, Thyme, Algae, Olive Leaf, to effectively clean the skin without stripping it dry throughout the day. The mechanisms of cleansing and antibacterial function are again achieved without any use of soaps, sulfates or synthetic chemicals of any sort. In this formula, waxes and debris in the skin are emulsified and lifted away without water rinsing.
3. Day & Night combination Spot Treatment: is a combination day/night spot treatment system that combines aspects of the former formulas and concentrates—with the addition of key ingredients for higher intensity response in localized areas on the skin. This product specifically is applied with a doe-foot wand; this too, is a first-to-market concept for Acne Spot Treatments. There are two components, one contains the day formula, one the night. The day formula is a clear liquid base that applies invisible to skin—similar to formula #2, however not the same. It is applied directly over an existing pimple or applied wherever a one feels a pimple may be forming under the surface of the skin. The complimentary Night treatment—similar to formula #1, however not the same—is applied at night and is opaque in appearance as it contains a similar combination of clays as the cleanser/mask.

All three of the aforementioned formulas can be used complimentary to, or successively of, each other. Additionally, all can be effectively utilized independently of each other. Efficacy is not determined by the use of all three.

Adsorbent cleansing: A blend of clays: Fullers Earth, Kaolin, Bentonite (ratios listed below) and Xanthan Gum are combined to cleanse the skin, pores, and pilo-sebaceous follicles through adsorbent cleansing. Through adsorbent cleansing, dirt, makeup, toxins, environmental debris, are molecularly bound to the formula and are lifted away when thoroughly rinsed. There are no soaps, or soap derivatives utilized in this formula. By binding microscopic debris through absorbency, there is a dramatic departure from using soap based products for cleansing, in that soaps tend to strip-away the acid mantle and lipid barrier and leave a residue behind.
Wax removal. The skin collects a waxy substance much like that of the outer ear canal. This substance can be exceptionally difficult to break down and is not emulsified with typical sulfates found in all Acne cleansing systems. Through the use of juices this waxy substance is not only broken up, it is drawn away from the skin and effectively cleared from the areas where it can clog the pores providing a breeding ground for bacteria, fungi and the like.
Skin Conditioning. The skin is simultaneously conditioned with a blend of Plant juices and extracts. This conditioning. The skin is simultaneously conditioned with a blend of Plant juices and extracts. It balances the color of the skin tone making it more even in appearance.
Anti-inflammation/Redness Reduction: Skin's overall inflammatory response is decreased by the use Arnica, Chamomile, Lavender and Hemp Seed oil. Reducing redness is critical to the overall appearance of healthy skin. Additionally, it reduces one of the key appearance issues that Acne suffers experience—that is the inflamed redness, plaguing millions.
Antibacterial/Antifungal: This is addressed not only through the FDA/OTC approved sulfur colloid, it is made more potent through the use of raw, organic sources of Oregano, Tea Tree, Golden Seal, Olive Leaf all of which display significant topical efficacy in reducing bacteria count and reducing the infection rate of Acne Sufferers.
Reduced Recurrence/Maintenance: Not only are the formulas effective at reducing and relieving the Acne sufferer of current outbreaks, they are very effective at reducing the frequency and intensity of recurring Acne lesions and redness. The mechanism for this is the overall synergistic effect of the herbal, vegetable and fruit combinations that help maintain healthy dermal tissues. Once initial clearing of skin has occurred a suggested use for maintenance is recommended. In addition, there are brand extensions in this specific category to address skin brightening, scar reduction, circulation, reduced inflammation.

### Specific Blends

### Fullers Earth, Kaolin, Bentonite, Xanthan Gum.

### Oregano, Olive Leaf, Thyme, Honeysuckle.

### Lemon, Lavender, Bergamot, Rosemary.

### Aloe, White Willow Bark, Arnica, Witch Hazel.

### Tea Tree, Golden Seal, Grapefruit Seed, Ylang Ylang.

(see formulas for ratios, ranges and applications)

### Example 2

### Acne Cleanser/Mask INCI and Basic Formulation

Sulfur Colloid: Active Ingredient

Fullers Earth

Kaolin Clay

Bentonite Clay

Xanthan Gum powder

Salix Alba (Willow) Bark Extract

Cananga Odorata (Ylang Ylang) Flower Oil

Lavandula Angustifolia (Lavender) Oil

Medica Limonum (Lemon) Peel Oil

Citrus Aurantium Bergamia (Bergamot) Fruit Oil

Rosmarinus Officinalis (Rosemary) Leaf Extract

Origanum Vulgare (Oregano) Leaf Extract

Thymus Vulgaris (Thyme) Extract

Olea europaea (Olive) Leaf Extract

Hydrastis canadensis (Golden Seal) Root Extract

Calendula Officinalis Flower Extract

Arnica Montana Flower Extract

### Phase A: Hydrate & Shear

2-4% Bentonite—completely hydrate with high speed shear and wait 30 minutes before next phase.

### Phase B: Homogenize

54-56% H2O,

16-18% Fullers Earth Pur Flo B80

14-16% Kaolin Clay (cosmetic grade)

Homogenize phase B into phase A and add:

0.2-0.4% Xanthan Gum during homogenization. Complete till smooth with no detectable particles.

Set-up another 30 minutes.

### Phase C: Blend in

5-7% Sulfur Colloid (100% Natural Active Ingredient)

1-2% White Willow Bark [all organic sources from this listing down]

0.5-1% Arnica

0.25-0.5% Rosemary

0.1-0.2% Oregano

0.1-0.2% Thyme

0.1-0.2% Olive

0.25-0.5% Golden Seal

1-2% Calendula

0.5-1% Arnica Montana

Phase D: Blend the Following Separately and in the Order Listed; then, Homogenize with Phase A-C

0.1-0.3% Ylang Ylang Flower Oil

0.5-0.7% Lavender Oil

0.1-0.3% Lemon Peel Oil

0.6-0.8% Bergamot Fruit Oil

Complete Scale-up and manufacturing with elected preservation system and requisite microbiology tests.

### Preservative System

An elected proprietary 100% natural broad-spectrum preservative system that effectively suppresses bacteria, molds and yeasts. Such as: Japanese Honeysuckle, Omegapien (100% natural pure plant oils of Oregano, Olive, Thyme, Rosemary plus Grapefruit Seed Extract). Phenethyl Alcohol, or other equally effective natural products.

The preservation system TBD once final product profile is complete for marketer. Overall fragrance, color and viscosity of final packaged product will determine which 100% Natural system of preservation is best utilized. Fragrance, Color and Viscosity are all determined by ratios of flower and fruit oils that are utilized in the product. There are no synthetic fragrances or colors used for adjustment; consequently, very slight and imperceptible differences in color and fragrance may occur between batches.

The embodiments discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A composition for the treatment of acne, comprising an effective amount of sulfur colloid and a 100% natural base.
